(19) United States
(12) Patent Application Publication
(10) Pub. No.: US 2010/0150944 A1
(43) Pub. Date: Jun. 17, 2010

(54) METHODS AND COMPOSITIONS FOR DIAGNOSIS AND TREATMENT OF DEPRESSION AND ANXIETY

(75) Inventors: Brian S. Hilbush, San Diego, CA (US); Peter B. Hedlund, San Diego, CA (US); Floyd E. Bloom, San Diego, CA (US); J. Gregor Sutcliffe, Cardiff, CA (US)

Correspondence Address: Casimir Jones, S.C., 2275 DEMING WAY, SUITE 310, MIDDLETON, WI, 53562, US

(73) Assignee: MODGENE, LLC, Cardiff by the Sea, CA (US)

(21) Appl. No.: 12/596,427

(22) PCT Filed: Apr. 15, 2008
(86) PCT No.: PCT/US08/60332

§ 371 Date: Feb. 11, 2010

Related U.S. Application Data
(60) Provisional application No. 60/923,627, filed on Apr. 16, 2007.

(51) Int. Cl. A61K 39/395; A61K 31/7052; A61K 31/352; A61K 36/906; A61K 31/519; A61K 31/5513; A61K 49/00; C12Q 1/48; G01N 33/68; A61P 25/24; A61P 25/22

(52) U.S. Cl. 424/172.1; 514/44 A; 514/456; 424/756; 514/249; 514/221; 800/3; 435/15; 436/86

(57) ABSTRACT

The present invention relates to identification of cellular components, genotypes and gene expression profiles associated with mood disorders. In some embodiments, the present invention relates to the correlation between ribosomal protein S6 (RPS6) and depression and/or anxiety. Embodiments of the present invention further relate to regulation of the activity of RPS6, e.g., by p90 Ribosomal S6 protein kinase. Embodiments of the present invention provide methods and compositions for, e.g., diagnosing, treating, and monitoring depression and/or anxiety, or risk thereof, and for selecting, monitoring, and tailoring treatments for depression and/or anxiety.

This application claims priority to U.S. Provisional Application Ser. No. 60/923,627, filed Apr. 16, 2007, which is incorporated herein by reference.

### FIELD OF THE INVENTION

The present invention relates to compositions and methods for diagnosis and treatment of depression and anxiety. In particular, the present invention provides compositions comprising an inhibitor of p90 ribosomal protein kinase and methods of using the same (e.g., as a therapeutic and/or prophylactic treatment for depression and/or anxiety). The present invention further comprises methods comprising assessing p90 ribosomal protein kinase (RSK) and ribosomal protein S6 (RPS6) activities (e.g., for diagnosis and for screening therapeutic agents for depression and/or anxiety).

### BACKGROUND OF THE INVENTION

Major depression is a clinical disorder with estimated lifetime prevalence as high as 17% (Fava M, Kendler K S. Neuron 28:335-341, 2000), making it one of the most common medical conditions in developed countries. Major depression is characterized by feelings of intense sadness and despair, mental slowing and loss of concentration, pessimistic worry, agitation, and self-deprecation. Physical changes also occur, especially in severe or “melancholic” depression. These include insomnia or hypersomnia, anorexia and weight loss (or sometimes overeating), decreased energy and libido, and disruption of normal circadian rhythms of activity, body temperature, and many endocrine functions. The etiology of depression is widely believed to involve the monoaminergic systems of the brain, and in particular the serotonergic system (Yadid G, Nakash R, Deri I, Tamar G, Kinar N, Gispan I, Zangen A. Prog Neurobiol 62:353-378, 2000; Adrien J. Sleep Med Rev 6:341-351, 2002). Treatment regimens commonly include the use of tricyclic antidepressants, monoamine oxidase inhibitors, some psychotropic drugs, lithium carbonate, and electroconvulsive therapy (see R. J. Baldessarini in Goodman & Gilman's The Pharmacological Basis of Therapeutics, 9th Edition, Chapter 19, McGraw-Hill, 1996 for a review). More recently, new classes of antidepressant drugs are being developed, including selective serotonin (5-HT) reuptake inhibitors (SSRIs), specific monoamine reuptake inhibitors and 5-HT1A receptor agonists, antagonists and partial agonists.

Anxiety is an emotional condition characterized by feelings such as apprehension and fear, accompanied by physical symptoms such as tachycardia, increased respiration, sweating and tremor. It is a normal emotion, but when it is severe and disabling, it becomes pathological. Anxiety disorders are generally treated using benzodiazepine sedative-antianxiety agents. Potent benzodiazepines are effective in panic disorder as well as in generalized anxiety disorder; however, the risks associated with drug dependency may limit their long-term use. 5-HT1A receptor partial agonists also have useful anxiolytic and other psychotropic activity, and less likelihood of sedation and dependence (see R. J. Baldessarini in Goodman & Gilman's The Pharmacological Basis of Therapeutics, 9th Edition, Chapter 18, McGraw-Hill, 1996 for a review).

Although antidepressant medication is successful in a majority of patients, many aspects need improvement. Some patients experience side effects or do not respond to treatment with currently available medications. With most antidepressants, the onset of clinical effect is delayed. The currently approved antidepressant drugs all target receptor sites of monoaminergic transmitter mechanisms, either slowing catabolism by inhibiting the degradative enzyme Monoamine Oxidase A or B, or by inhibiting the neurotransmitter transporters for norepinephrine or serotonin or both. While these medications are effective in approximately 40% of patients with the diagnosis of major depressive disorder, the therapeutic effects are slow to emerge (10-14 days) even though the doses given quickly inhibit their targets. Many of the early, norepinephrine-only transporter inhibitors produced significant sedation, induced seizures and led to significant cardiac toxicity, and led to the development of serotonin-selective transporter inhibitors with significantly less cardiotoxicity, but those drugs have been found to produce variable sedation, weight gain, and sexual dysfunction. A recently studied combination norepinephrine and serotonin reuptake inhibitor causes very significant withdrawal symptoms, and that becomes very problematic since compliance with medications chronically is typically poor.

What is needed are medications that would act quickly and selectively to relieve the negative emotional status, inability to concentrate, changes in appetite and sleep, decreased interest in pleasurable stimuli, and recurrent thoughts of death, despair and suicide, quickly and with minimal side effects in central arousal, seizure threshold, or arousal, and that would be directed towards the specific brain mechanisms that underlie the vulnerability to react to stressful environmental stimuli symptomatically with depression. The development of new medications to overcome these deficiencies will require a deeper understanding of the molecular mechanisms that underlie depression and its response to medication.

### SUMMARY OF THE INVENTION

The present invention relates to identification of cellular components, genotypes and gene expression profiles associated with mood disorders. In some embodiments, the present invention relates to the correlation between ribosomal protein S6 (RPS6) and depression and/or anxiety. Embodiments of the present invention further relate to regulation of the activity of RPS6, e.g., by p90 Ribosomal S6 protein kinase, and methods and compositions for, e.g., diagnosing, treating, and monitoring depression and/or anxiety, or risk thereof.

In some embodiments, the present invention provides a method of treating or preventing depression or anxiety in a subject comprising administering a pharmaceutical preparation comprising a therapeutic agent that alters the state of ribosomal protein S6 (RPS6) in a cell in said subject. In some embodiments, the altering of the state of the ribosomal protein S6 comprises altering the activity of a protein kinase, e.g., a protein kinase that acts upon RPS6. In some embodiments, the present invention provides a method of treating or preventing depression or anxiety in a subject comprising administering a pharmaceutical preparation comprising a therapeutic agent that alters the activity of a protein kinase in a cell in the subject. In some preferred embodiments, the protein kinase is p90 Ribosomal S6 Kinase. In some particularly preferred embodiments, the altering of the activity of the protein kinase comprises inhibiting the protein kinase.

In some embodiments, the therapeutic agent comprises an inhibitor of p90 Ribosomal S6 Kinase. In some preferred embodiments, the therapeutic agent comprises a specific inhibitor of p90 Ribosomal S6 Kinase.

In some embodiments, the therapeutic agent comprises an antisense oligonucleotide, while in some embodiments, the therapeutic agent comprises an interfering RNA. In yet other embodiments, the therapeutic agent comprises an aptamer.

In some embodiments, the therapeutic agent comprises an antibody, while in some embodiments, said therapeutic agent comprises a small molecule. In some preferred embodiments, the therapeutic agent comprises a flavenoid extract of Forsteronia refracta or Zingiber zerumbet.

In some embodiments, the therapeutic agent comprises an SL0101 compound or a pharmaceutically acceptable salt or hydrate thereof. In some preferred embodiments, the SL0101 compound comprises SL0101-1, SL0101-2 or SL0101-3, or 3Ac-SL0101. In some embodiments, therapeutic agent comprises a chemical derivative, a chemical analog, or a chemical precursor of a SL0101 compound, or a pharmaceutically acceptable salt or hydrate thereof.

In some embodiments, the therapeutic agent comprises a pteridinone composition, e.g., a chemical derivative, a chemical analog, or a chemical precursor of a pteridinone (see, e.g., Denny, et al., WO0119825A1, which is incorporated herein by reference). In some preferred embodiments, the therapeutic agent comprises a dihydropteridinone composition, e.g., a chemical derivative, a chemical analog, or a chemical precursor of a dihydropteridinone (see, e.g., U.S. Pat. Nos. 7,332,491 and 6,806,272, each of which is incorporated herein by reference). In some particularly preferred embodiments, the therapeutic agent comprises a dihydropteridinone composition comprising B1-D1870 or a pharmaceutically acceptable salt or hydrate thereof.

In some embodiments of the present invention, the pharmaceutical preparation comprises a composition as described above, and further comprises a second therapeutic agent. In some embodiments, the second therapeutic is a known therapeutic agent for treatment of depression. In some preferred embodiments, the known therapeutic for the treatment of depression is selected from the group consisting of a selective serotonin reuptake inhibitor, a serotonin and norepinephrine reuptake inhibitor, a dopamine reuptake inhibitor, a tetracyclic antidepressant, a combined reuptake inhibitor, a receptor blocker, tricyclic antidepressant, and a monoamine oxidase inhibitor. In particularly preferred embodiments, the known therapeutic for the treatment of depression is selected from the group consisting of citalopram, escitalopram, fluoxetine, paroxetine, sertraline, duloxetine, venlafaxine, bupropion, mirtazapine, trazodone, tefazodone, maprotiline, amitriptyline, amoxapine, desipramine, doxepin, imipramine, nortriptyline, protriptyline, trimipramine, phenelzine, tranylcypromine, isocarboxazid, and selegilin.

In some embodiments, the second therapeutic is a known therapeutic agent for treatment of anxiety. In some preferred embodiments, the known therapeutic for the treatment of anxiety is selected from the group consisting of a benzodiazepine, a beta-blocker, and a non-benzodiazepine hypnotic. In some particularly preferred embodiments, the known therapeutic agent for the treatment of anxiety is selected from the group consisting of diazepam, nitrazepam, alprazolam, bromazepam, chlordiazepoxide, chlorazepate, lorazepam, oxazepam, flunitrazepam, flurazepam, loprazolam, lormetazepam, and temazepam, buspirone, meprobamate, zalepon, zolpidem, zopiclone, chloral hydrate, triclofos, clomethizole, and meprobamate.

The present invention is not limited to any particular preparation. For example, in some embodiments, the pharmaceutical preparation comprising a therapeutic agent, described above, comprises a powder, a granule, a suspension, a solution, a capsule, a sachet, a lozenge, or a tablet.

The present invention is not limited to any particular route of administration. In some embodiments, the administering of the pharmaceutical preparation comprising a therapeutic agent, described above, comprises oral administration, while in some embodiments, the administering comprises topical administration. In some embodiments, the topical administration comprises transdermal administration. In some embodiments, the administering comprises parenteral administration, while in some preferred embodiments, the parenteral administration comprises intravenous, intraarterial, subcutaneous, intraperitoneal, intramuscular, intracranial, intrathecal, or intraventricular injection or infusion. In some embodiments, the administering comprises pulmonary administration.

In some embodiments, the present invention provides a method of selecting a compound for treatment or prevention of depression or anxiety, comprising selecting a test compound suspected of altering the state of ribosomal protein S6; and determining the ability of said test compound to inhibit despair behavior in a test animal in a despair-inducing test, wherein inhibition of despair behavior is indicative of efficacy of said test compound in the treatment or prevention of depression or anxiety. In some embodiments, the test animal is a mouse and the despair-inducing test is selected from the group consisting of a forced swim test and a tail suspension test. In some preferred embodiments, the despair behavior is immobility. In some embodiments, the method of selecting a compound for treatment or prevention of depression or anxiety further comprises determining whether the test compound reduces despair behavior in a dose dependent manner. In some embodiments, the test animal is a member of a strain of recombinant inbred mice, and the dose-dependent manner comprises dose-dependent suppression of immobility in proportion to the expression in the brain of the strain of mice of a protein kinase known to phosphorylate ribosomal protein S6. In some embodiments, the protein kinase comprises a p90 ribosomal protein kinase.

In some embodiments, the present invention provides a method of screening a compound for use in treatment or prevention of depression or anxiety, comprising determining the effect of the compound on the state of ribosomal protein S6, wherein alteration of the state of said ribosomal protein S6 is indicative of utility of the compound in treatment or prevention of depression or anxiety.

In some embodiments, the present invention provides a method of screening a compound for use in treatment or prevention of depression or anxiety, comprising determining the effect of the compound on the activity of a p90 ribosomal protein kinase, wherein alteration of the activity of the p90 ribosomal protein kinase is indicative of utility of said compound in treatment or prevention of depression or anxiety. In some preferred embodiments, the alteration of the activity of the p90 ribosomal protein kinase comprises inhibition of the kinase. In some preferred embodiments, the method comprises use of an in vitro assay for protein kinase activity.

In some embodiments of the method of screening described above, the protein kinase activity is an activity of a ribosomal protein kinase. In particularly preferred embodiments, the ribosomal protein kinase is specific for phosphorylation of ribosomal protein S6.

In some embodiments, the present invention provides a method of screening a subject for depression or anxiety, comprising providing a sample from the subject and determining a feature of ribosomal protein S6 in said sample, wherein the feature of ribosomal protein S6 is indicative of the presence or risk of developing depression or anxiety. In some preferred embodiments, determining the feature of ribosomal protein S6 comprises measuring the concentration of ribosomal protein S6 in the sample, while in some embodiments, determining a feature of comprises measuring an amount of ribosomal protein S6 mRNA. In some embodiments, determining a feature of ribosomal protein S6 comprises determining a feature of nucleic acid encoding ribosomal protein S6. In some embodiments, determining a feature of a gene comprises determining the presence or absence of a sequence (e.g., a mutation or polymorphism, including but not limited to a single nucleotide polymorphism), associated with expression or function (e.g., transcription, translation, protein function, etc.) of ribosomal protein S6. Sequences need not be in the coding region of a ribosomal protein S6 gene, however. It is contemplated that polymorphisms in non-coding regions (e.g., flanking regions, introns, etc.) associated with ribosomal protein S6 function find use in the present invention.

In some embodiments, the present invention provides a method of screening a subject for depression or anxiety, comprising providing a sample from a subject, and determining an activity of p90 ribosomal protein kinase in the sample, wherein activity of ribosomal protein S6 is indicative of the presence or risk of developing depression or anxiety. In some embodiments, determining the activity of p90 ribosomal protein kinase comprises determining the concentration of p90 ribosomal protein kinase protein in the sample, while in some embodiments, determining the activity of p90 ribosomal protein kinase comprises determining the phosphorylation activity of p90 ribosomal protein kinase in the sample on a substrate. In some embodiments, determining the activity of a p90 ribosomal protein kinase in a sample comprises measuring an amount of p90 ribosomal protein kinase mRNA. In some embodiments, determining the activity of a p90 ribosomal protein kinase comprises determining a feature of nucleic acid encoding p90 ribosomal protein kinase. In some embodiments, determining a feature of a gene comprises determining the presence or absence of a sequence (e.g., a mutation or polymorphism, including but not limited to a single nucleotide polymorphism), associated with expression or function (e.g., transcription, translation, protein function, etc.) of p90 ribosomal protein kinase. Sequences need not be in the coding region of a p90 ribosomal protein kinase gene, however. It is contemplated that polymorphisms in non-coding regions (e.g., flanking regions, introns, etc.) associated with p90 ribosomal protein kinase function find use in the present invention.

In some embodiments, the present invention provides methods of tailoring treatments to the biochemical status of a subject or patient. It is contemplated that features of ribosomal protein S6 and/or p90 ribosomal protein kinase may be altered by particular biochemical circumstances of a subject or patient, including but not limited to the presence of other drugs or medications (e.g. for treatment of depression or unrelated conditions), or biochemical changes caused by other circumstances. The present invention provides methods for determining features of ribosomal protein S6 and/or p90 ribosomal protein kinase as described herein, which find use for monitoring the effect of the biochemical status of the subject on ribosomal protein S6 and/or p90 ribosomal protein kinase. In some embodiments, therapy for depression and/or anxiety is selected, adjusted, or altered accordingly.

In some embodiments, the sample comprises tissue from the subject. In preferred embodiments, the tissue comprises neuronal tissue, and in particularly preferred embodiments, the tissue comprises brain tissue. In some embodiments, the sample comprises a fluid from the subject. In preferred embodiments, the fluid comprises cerebrospinal fluid.

In some embodiments, the present invention provides a composition comprising a pharmaceutical preparation of a purified kinase inhibitor and a second therapeutic agent. In some embodiments, the second therapeutic is a known therapeutic agent for treatment of depression. In some preferred embodiments, the known therapeutic for the treatment of depression is selected from the group consisting of a selective serotonin reuptake inhibitor, a serotonin and norepinephrine reuptake inhibitor, a dopamine reuptake inhibitor, a tetracyclic antidepressant, a combined reuptake inhibitor, a receptor blocker, a tricyclic antidepressant, and a monoamine oxidase inhibitor. In particularly preferred embodiments, the known therapeutic for the treatment of depression is selected from the group consisting of citalopram, escitalopram, fluoxetine, paroxetine, sertraline, duloxetine, venlafaxine, bupropion, mirtazapine, trazodone, tefazodone, maprotiline, amitriptyline, amoxapine, desipramine, doxepin, imipramine, nortriptyline, protriptyline, trimipramine, phenelzine, tranylcypromine, isocarboxazid, and selegilin.

In some embodiments of the pharmaceutical preparation of a purified kinase inhibitor and a second therapeutic agent, the second therapeutic is a known therapeutic agent for treatment of anxiety. In some preferred embodiments, the known therapeutic for the treatment of anxiety is selected from the group consisting of a benzodiazepine, a beta-blocker, and a non-benzodiazepine hypnotic. In some particularly preferred embodiments, the known therapeutic agent for the treatment of anxiety is selected from the group consisting of diazepam, nitrazepam, alprazolam, bromazepam, chlordiazepoxide, chlorazepate, lorazepam, oxazepam, flunitrazepam, flurazepam, loprazolam, lormetazepam, and temazepam, buspirone, meprobamate, zalepon, zolpidem, zopiclone, chloral hydrate, triclofos, clomethizole, and meprobamate.

While not limiting the preparation to any particular composition or form, in some embodiments, the pharmaceutical preparation comprises a powder, a granule, a suspension, a solution, a capsule, a sachet, a lozenge, a tablet, a transdermal patch, an ointment, a lotion, a cream, a gel, a drop, a suppository, a spray, a foam, or an aerosol.

Particular embodiments of the invention are described in this Summary, and below, in the Brief and Detailed descriptions of the invention. Although the invention has been described in connection with specific embodiments, it should be understood that the invention as claimed should not be unduly limited to such specific embodiments.

### DESCRIPTION OF THE DRAWINGS

FIG. 1 shows a graph comparing the rpS6 mRNA concentrations in the brains of C57BL/6J and DBA/2J mice.

FIG. 2 compares the times of immobility of C57BL/6J and DBA/2J mice in a forced swim test, either untreated, or after treatment with a kinase inhibitor in accordance with the present invention.

FIG. 3 shows a graph indicating the dose response of C57BL/6J mice treated with varying dosages of a kinase inhibitor prior to forced swim testing.

FIG. 4 compares the times of immobility of C57BL/6J mice in a forced swim test, either untreated, or after treatment with a kinase inhibitor B1-D1870.

### DEFINITIONS

As used herein, the use of the term “RSK” is intended to refer the family of p90 ribosomal S6 kinase isotypes, including RSK1, RSK2, RSK3 and RSK4. RSK1, RSK2, RSK3 and RSK4 are specific human isotypes that have previously been described in the literature. The nucleic acid and protein sequences of these isotypes are found at Genbank accession numbers NM.sub.—002953 (for RSK1), NM 004586 (for RSK2), NM.sub.—021135 (for RSK3;) and NM.sub.—014496 (for RSK4).

As used herein, the term “RSK inhibitor” or “ribosomal protein kinase inhibitor” includes any compound or condition that specifically inhibits or reduces the kinase activity of RSK or which inhibits any function of RSK, either directly or indirectly. Such inhibitory effects may result, e.g., from directly or indirectly interfering with the protein's ability to phosphorylate its substrate, may result from inhibiting the expression (transcription and/or translation) of RSK, or may result by interfering with or altering subcellular localization (e.g., nuclear, membrane, cytosolic) of RSK and/or its substrate.

As used herein, the term “RSK specific inhibitor” or “p90 ribosomal protein kinase specific inhibitor” includes any compound or condition that inhibits RSK kinase activity, directly or indirectly, (including any or all of the individual RSK isotypes) without substantially impacting the activity of other kinases. Such inhibitory effects may result, e.g., from directly or indirectly interfering with the protein's ability to phosphorylate its substrate, or may result from inhibiting the expression (transcription and/or translation) of RSK, or may result by interfering with or altering subcellular localization (e.g., nuclear, membrane, cytosolic) of RSK and/or its substrate.

As used herein, the term “alters the state” as used in reference to alteration of a protein refers to a change in a protein or a component of a protein from one state or status to another (e.g., unphosphorylated to phosphorylated; oxidized to reduced, a first conformation to a second conformation; unactivated to activated, unbound to bound, etc., including the inverses thereof).

As used herein, the term “alters a feature” as used in reference to alteration of a protein refers, without limitation, to any change in a physical or activity feature (e.g., amino acid or sequence of amino acids, conformation, availability of a binding pocket or other interactive element, addition or removal of a chemical group (e.g., a phosphate), etc. Alteration of a feature of a protein may entail, but does not require, alteration of the state of the protein.

As used herein, the terms “host,” “subject” and “patient” refer to any animal, including but not limited to, human and non-human animals (e.g., dogs, cats, cows, horses, sheep, poultry, fish, crustaceans, etc.) that is studied, analyzed, tested, diagnosed or treated. As used herein, the terms “host,” “subject” and “patient” are used interchangeably, unless indicated otherwise.

As used herein, the terms “subject having depression” or “subject displaying signs or symptoms or pathology indicative of depression” or “subjects suspected of displaying signs or symptoms or pathology indicative of depression” refer to a subject that is identified as having or likely to have depression based on known depression signs, symptoms and pathology.

As used herein, the terms “subject at risk of displaying pathology indicative of depression” and “subject at risk of depression” refer to a subject identified as being at risk for developing depression.

As used herein, the term “antidepressant” refers to an agent used to treat or prevent depression. Such agents include, but are not limited to, small molecules, drugs, antibodies, pharmaceuticals, and the like.

As used herein, “anxiolytic” refers to an agent used to treat or prevent anxiety. Such agents include, but are not limited to, small molecules, drugs, antibodies, pharmaceuticals, and the like.

As used herein, the terms “subject having anxiety” or “subject displaying signs or symptoms or pathology indicative of anxiety” or “subjects suspected of displaying signs or symptoms or pathology indicative of anxiety” refer to a subject that is identified as having or likely to have anxiety based on known anxiety signs, symptoms and pathology.

As used herein, the terms “subject at risk of displaying pathology indicative of anxiety” and “subject at risk of anxiety” refer to a subject identified as being at risk for developing anxiety.

As used herein, the term “cognitive function” generally refers to the ability to think, reason, concentrate, or remember. Accordingly, the term “decline in cognitive function” refers to the deterioration of lack of ability to think, reason, concentrate, or remember.

The terms “sample” and “specimen” are used in their broadest sense and encompass samples or specimens obtained from any source. As used herein, the term “sample” is used to refer to biological samples obtained from animals (including humans), and encompasses fluids, solids, tissues, and gases. In some embodiments of the invention, biological samples include neural tissue (e.g., brain tissue) cerebrospinal fluid (CSF), serous fluid, urine, saliva, blood, and blood products such as plasma, serum and the like. However, these examples are not to be construed as limiting the types of samples that find use with the present invention.

As used herein, the term “effective amount” refers to the amount (e.g., of a composition comprising a kinase inhibitor of the present invention) sufficient to produce a selected effect. For example, an effective amount of an RSK inhibitor is an amount of the inhibitor sufficient to reduce RSK activity, as determined, e.g., by observation of an in vivo effect associated with reduced RSK activity, or by use of an in vitro assay such as serine/threonine kinase assay. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment (e.g., compositions of the present invention) to a subject (e.g., a subject or in vivo, in vitro, or ex vivo cells, tissues, and organs). Exemplary routes of administration to the human body can be through the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the term “treating” includes administering therapy to prevent, cure, or alleviate/prevent the symptoms associated with, a specific disorder, disease, injury or condition.

As used herein, the terms “co-administration” and “co-administering” refer to the administration of at least two agent(s) (e.g., compositions comprising a kinase inhibitor and one or more other agents—e.g., a depression disease therapeutic) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “treatment” or grammatical equivalents encompasses the improvement and/or reversal of the symptoms of disease (e.g., depression). A compound which causes an improvement in any parameter associated with disease when used in the screening methods of the instant invention may thereby be identified as a therapeutic compound. The term “treatment” refers to both therapeutic treatment and prophylactic or preventative measures. For example, those who may benefit from treatment with compositions and methods of the present invention include those already with a disease and/or disorder (e.g., depression, anxiety, or symptoms or pathologies consistent with depression and/or anxiety) as well as those in which a disease and/or disorder is to be prevented (e.g., using a prophylactic treatment of the present invention).

As used herein, the term “at risk for disease” refers to a subject (e.g., a human) that is predisposed to experiencing a particular disease. This predisposition may be genetic (e.g., a particular genetic tendency to experience the disease, such as heritable disorders), or due to other factors (e.g., age, weight, environmental conditions, exposures to detrimental compounds present in the environment, etc.). Thus, it is not intended that the present invention be limited to any particular risk, nor is it intended that the present invention be limited to any particular disease.

As used herein, the term “suffering from disease” refers to a subject (e.g., a human) that is experiencing a particular disease. It is not intended that the present invention be limited to any particular signs or symptoms, nor disease. Thus, it is intended that the present invention encompasses subjects that are experiencing any range of disease (e.g., from sub-clinical manifestation to full-blown disease) wherein the subject exhibits at least some of the indicia (e.g., signs and symptoms) associated with the particular disease.

As used herein, the terms “disease” and “pathological condition” are used interchangeably to describe a state, signs, and/or symptoms that are associated with any impairment of the normal state of a living animal or of any of its organs or tissues that interrupts or modifies the performance of normal functions, and may be a response to environmental factors (such as emotional trauma, physical trauma, malnutrition, industrial hazards, or climate), to specific infective agents (such as worms, bacteria, or viruses), to inherent defect of the organism (such as various genetic anomalies, or to combinations of these and other factors.

The term “compound” refers to any chemical entity, pharmaceutical, drug, and the like that can be used to treat or prevent a disease, illness, sickness, or disorder of bodily function. As used herein, a compound may be a single composition (e.g., a pure preparation of a chemical) or it may be a composition comprising a plurality of chemicals (e.g., one or more effective agents and one or more inert agents). A compound may comprise both known and potential therapeutic compositions. A compound can be determined to be therapeutic by screening using the screening methods of the present invention.

A “known therapeutic” compound or agent includes a therapeutic compound that has been shown (e.g., through animal trials or prior experience with administration to humans) to have a therapeutic effect in a treatment. However, a known therapeutic compound is not limited to a compound having a particular level of effectiveness in the treatment or prevention of a disease (e.g., depression or anxiety). Examples of known antidepressant therapeutic agents include, but are not limited to, selective serotonin reuptake inhibitors (SSRIs, e.g., citalopram (CELEXA), escitalopram (LEXAPRO), fluoxetine (PROZAC, PROZAC WEEKLY), paroxetine (PAXIL, PAXIL CR) and sertraline (ZOLOFT); serotonin and norepinephrine reuptake inhibitors (SNRIs, e.g., duloxetine (CYMBALTA) and venlafaxine (EFFEXOR, EFFEXOR XR); norepinephrine and dopamine reuptake inhibitors (NDRIs, e.g., bupropion (WELLBUTRIN, WELLBUTRIN SR, WELLBUTRIN XL); tetracyclic antidepressants (e.g., mirtazapine (REMERON, REMERON SOLTAB); combined reuptake inhibitors and receptor blockers (e.g., trazodone, tefazodone, maprotiline); tricyclic antidepressants (TCAs, e.g., amitriptyline, amoxapine, desipramine (NORPRAMIN), doxepin (SINEQUAN), imipramine (TOFRANIL), nortriptyline (PAMELOR), protriptyline (VIVACTIL), trimipramine (SURMONTIL)); monoamine oxidase inhibitors (MAOIs, e.g., phenelzine (NARDIL), tranylcypromine (PARNATE), isocarboxazid (MARPLAN), and selegiline (EMSAM)). Examples of known anxiolytic therapeutic agents include, but are not limited, benzodiazepines (e.g., diazepam (VALIUM), nitrazepam (MOGADON), alprazolam (XANAX), bromazepam (LEXOTAN), chlordiazepoxide (LIBRIUM), chlorazepate (TRANXENE), lorazepam (ATIVAN), oxazepam, flunitrazepam (ROHYPNOL), flurazepam (DALMANE), loprazolam, lormetazepam, and temazepam); non-benzodiazepine agents (e.g., buspirone (BUSPAR), beta-blockers, and meprobamate (EQUAGESIC)); and non-benzodiazepine hypnotics (e.g., zalepon (SONATA), zolpidem (STILLNOCT), zopiclone (ZIMOVANE), chloral hydrate, triclofos, and clomethizole).

As used herein, the term “small molecule” generally refers to a molecule of less than about 10 kDa molecular weight, including but are not limited to natural or synthetic organic or inorganic compounds, peptides, (poly)nucleotides, (oligo)saccharides and the like. Small molecules specifically include small non-polymeric (i.e., not peptide or polypeptide) organic and inorganic molecules.

As used herein the term “extract” and like terms refers to a process of separating and/or purifying one or more components from their natural source, or when used as a noun, refers to the composition produced by such a process.

As used herein, the term “kit” refers to any delivery system for delivering materials. In the context of kinase activity or inhibition assays, such delivery systems include systems that allow for the storage, transport, or delivery of reaction reagents and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains standards for comparison to test compounds. The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “toxic” refers to any detrimental or harmful effects on a subject, a cell, or a tissue as compared to the same cell or tissue prior to the administration of the toxicant.

As used herein, the term “pharmaceutically purified” refers to a composition of sufficient purity or quality of preparation for pharmaceutical use.

As used herein, the term “purified” refers to a treatment of a starting composition to remove at least one other component (e.g., another component from a starting composition (e.g., plant or animal tissue, an environmental sample etc.), a contaminant, a synthesis precursor, or a byproduct, etc.), such that the ratio of the purified component to the removed component is greater than in the starting composition.

As used herein, the term “pharmaceutical composition” refers to the combination of an active agent (e.g., composition comprising a kinase inhibitor) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. (1975), incorporated herein by reference.

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the terms “gene expression” and “expression” refer to the process of converting genetic information encoded in a gene into RNA (e.g., mRNA, rRNA, tRNA, or snRNA) through “transcription” of the gene (i.e., via the enzymatic action of an RNA polymerase), and, for protein encoding genes, into protein through “translation” of mRNA. Gene expression can be regulated at many stages in the process. “Up-regulation” or “activation” refer to regulation that increases and/or enhances the production of gene expression products (e.g., RNA or protein), while “down-regulation” or “repression” refer to regulation that decrease production.

Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., depression, anxiety, etc.). Test compounds comprise both known and potential therapeutic compounds. A test compound can be determined to be therapeutic by screening using the screening methods of the present invention.

As used herein, a “functional” molecule is a molecule in a form in which it exhibits a property by which it is characterized. By way of example, a functional enzyme is one which exhibits the characteristic catalytic activity by which the enzyme is characterized.

As used herein the term “antisense oligonucleotide” refers to a nucleic acid, e.g., an RNA or DNA segment, that is complementary to the sequence of a target RNA (or fragment thereof). Typically, the target RNA is a mRNA expressed by a cell.

As used herein the term “interfering oligonucleotide” relates to an oligonucleotide, generally an RNA oligonucleotide, capable of inhibiting the function of a target gene product. More particularly, the interfering oligonucleotide is a polynucleotide sequence that comprises a sequence identical or homologous to a target gene (or fragment thereof). There are two different types of interference RNA (RNAi), short interfering RNA (siRNA) and short hairpin RNA (shRNA). Short interfering RNAs typically consist of 19-22 nt double-stranded RNA molecules that can be chemically synthesized, or generated from larger (>100 nucleotide) double stranded RNA (dsRNA) by enzymatic cleavage using an RNase III-like enzyme called Dicer. Short hairpin RNA, consists of 19-29 nt palindromic sequences connected by loop sequences, that are prepared by chemical synthesis or through recombinant DNA techniques.

### DETAILED DESCRIPTION OF THE INVENTION

The present invention relates to identification of cellular components, genotypes and gene expression profiles associated with mood disorders. In some embodiments, the present invention relates to the correlation between ribosomal protein S6 (RPS6) and depression and/or anxiety. Embodiments of the present invention further relate to regulation of the activity of RPS6, e.g., by p90 Ribosomal S6 protein kinase. Embodiments of the present invention provide methods and compositions for, e.g., diagnosing, treating, and monitoring depression and/or anxiety, or risk thereof, and for selecting, monitoring, and tailoring treatments for depression and/or anxiety.

### Animal Models for Depression

Animal models have been developed that have been shown to have high value for predicting antidepressant activity of a drug. Two of the most-used models are the forced-swim (Porsolt R D, Bertin A, Jalfre M. Arch Int Pharmacodyn Ther 229:327-336, 1977) and the tail-suspension tests (Stem L, Chemat R, Thierry B, Simon P. Psychopharmacol 85:367-370, 1985). The forced-swim test was originally developed for rats, but has been shown to work equally well in mice (Cryan J F, Markou A, Lucki I Trends Pharmacol Sci 23:238-245, 2002), allowing the use of both tests to evaluate knockout mice (Cryan J F, Mombereau C. Mol Psychiatry 9:326-357, 2004). Thus, it has been shown that mice lacking the 5-HT1A receptor show antidepressant-related behavior (Heisler L K, Chu H M, Brennan T J, Danao J A, Bajwa P, Parsons L H, Tecott L H. Proc Natl Acad Sci 95:15049-15054, 1998; Parks C L, Robinson P S, Sibille E, Shenk T, Toth M. Proc Natl Acad Sci 95:10734-10739, 1998), whereas the results in mice lacking the 5-HT transporter are more inconclusive (Holmes A, Yang R J, Murphy D L, Crawley A. Neuropsychopharmacol 27:914-923, 2002). Recently the 5-HT7 receptor has been implicated in these model behaviors (Hedlund P B, Huitron-Resendiz S, Henriksen S J, Sutcliffe J G. Biol Psychiatry 58: 831-837, 2005).

Briefly, in the Porsolt forced swim test, swim sessions are conducted by placing animals in containers with a level of water selected to be deep enough so that the animal cannot touch the bottom with its hind limbs or tail, nor can it escape. Two swim sessions are conducted, an initial 15 minute pretest one day prior to administration of the antidepressant drug and a second 5 minute test after administration of the drug (e.g., infusion of the drug into the midbrain) is begun. Each animal's 5 minute test sessions are videotaped for scoring later. The amount of time the animal spends active (swimming, exploring or trying to escape) and the time the rat is immobile (not struggling and making only those movements necessary to keep its head above water) is measured. Drugs with anti-depressant like activity decrease the immobility time.

The Kasahara test is a modification of the above Porsolt test in that it employs a water wheel instead of a cylinder (Kasahara, et al., Life Sci., 52(22):1741-1749 1993; Nomura et al., Eur. J. Pharmacol. 83(3-4) 171-175, 1982). Essentially a water wheel is placed in a water tank. Mice are placed on the apparatus so that they run on the wheel making it turn. If the mice try and “escape” from the water, they turn the wheel vigorously, when the mice begin to despair, they stop running and the wheel stops turning. Thus the number of turns of the wheel is indicative of level of depression of the animal. A few hours before the test, the control and treatment groups of mice, respectively, receive subcutaneous injections of placebo or drug to be tested. The mice are individually placed in the swimming wheel and the number of turns of the wheel is recorded for each. Antidepressant activity is indicated by an increased number of turns in the treatment group.

An additional procedure for monitoring the effects of antidepressants involves suspending the mice by the tail from a lever and recording movements of the animal. The total duration of the test (usually about 6 minutes) can be divided into periods of agitation and immobility. Antidepressants will generally decrease the duration of immobility (Stem et al., Psychopharmacology 85(3) 367-70). A computerized version of this test, referred to as ITEMATIC-TST has been developed and is well known to those of skill in that art as a primary screening test for antidepressant activity (Stem et al., Prog. Neuropsychpharmacol. Biol. Psychiatry 11(6)659-671, 1987). Additional devices for monitoring depression also are known to those of skill in the art (Nomura et al., Yakubutsu Seishiiz Kodo 12(5)207-13, 1992).

In addition to the behavioral assays described above, those of skill in the art can monitor chronic stress-induced neurochemical changes. In such an assay, on would perform neurochemical measurements in which serotonin levels were monitored as a function of stress induction. Serotonin and its principle metabolite, 5-hydroxyindole-acetic acid (5-HIAA), can be measured using an isocratic HPLC elution system and electrochemical detection, using a 16 channel coulometric array detector according to methods well known to those of skill in the art (ESA, Inc., Bedford, Mass.; Gamache et al, Neurosci. Abstracts 17:985, 1991).

Significant changes in depression-related events used to characterize depression-like behavior of the animals include decreased levels of brain-derived neurotrophic factor mRNA or protein in the brain, especially in the hippocampus; increased levels of corticotrophin-releasing factor in then cerebrospinal fluid; increased levels or arginine vasopressin mRNA or protein in the paraventicular nucleus; increased hypothalamic pituitary axis activity; resistance to suppression of ACTH by dexamethasone; cognitive disturbances; or increased levels of corticosterone in serum. Anti-depressant therapy would be expected to reverse, at least partially, these changes.

### Response Differences Between Mouse Strains in Depression Testing

There are significant differences between mouse strains in the forced-swim and tail-suspension tests (Holmes A, Yang R J, Murphy D L, Crawley J N. Neuropsychopharmacol 27:914-923, 2002; Lucki I, Dalvi A, Mayorga A J. Psychopharmacol (Berl) 155:315-322, 2001; Liu X, Gershenfeld H K. Biol Psychiatry. 49:575-581, 2001; Trullas R, Jackson B, Skolnick P. Psychopharmacology (Berl). 99:287-288, 1989; van der Heyden J A, Molewijk E, Olivier B. Psychopharmacology (Berl). 92:127-130, 1987; Vaugeois J M, Passera G, Zuccaro F, Costentin J. Psychopharmacology (Berl). 134:387-391, 1997). For example, Lucki and colleagues (Lucki I, Dalvi A, Mayorga A J. Psychopharmacol (Berl) 155:315-322, 2001) studied 7 inbred and 4 outbred mouse lines in the forced-swim test. They found that, while there was considerable intrastrain variability both in the baseline immobility values and their response to antidepressant medication for the outbred lines, intrastrain variability for the inbred lines was very small. Different lines exhibited both different baseline responses and different drug responses. For example, during the last 4 minutes of a 6-minute forced swim, unmedicated C57BL/6J mice spent 143 seconds immobile; they responded to the tricyclic desipramine (10 mg/kg) by reducing immobility to 100 seconds, but did not show significant response to the SSRI fluoxetine (10 mg/kg), remaining immobile for 128 seconds. In contrast, DBA/2J mice had baseline immobilities of 100 seconds, and responded to both desipramine (50 seconds) and fluoxetine (50 seconds). Thus, genetic factors influence behaviors in the test. The authors discuss the possibility that some of these may affect serotonin metabolism and stress responses, factors that may be relevant to susceptibility to depression in humans.

El Yacoubi and colleagues (El Yacoubi M, Bouali S, Popa D, Naudon L, Leroux-Nicollet I, Hamon M, Costentin J, Adrien J, Vaugeois J M. Proc Natl Acad Sci USA 100:6227-6232, 2003) selectively bred mice for their immobility times in the tail-suspension test so as to produce lines that were “helpless” or non-helpless. The helpless mice were 40-fold more immobile than the non-helpless line in the tail-suspension test, and 2.5-fold more immobile in the forced swim, indicating that these tests measure overlapping, but not identical, behavioral responses. Interestingly (because in humans females are more susceptible to depression than are males), the female helpless mice were more immobile than males in both tests, suggesting that endocrinological factors may interact with other susceptibility mechanisms. The helpless mice spent less time awake, and more time in slow-wave and REM sleep, with a decrease in REM latency and increased sleep fragmentation, as well as lower 5-HT metabolic index and higher corticosterone levels, all features characteristic of human depression. These observations suggest that the two animal tests represent models with pertinence to the study of depression, even though they are conducted after acute administration of antidepressant compounds that, in humans, require chronic administration for clinical benefit. The two studies suggest that genetic factors, hence the activities of the protein products of genes, influence behaviors in the models.

### Genetic Factors in Strain Differences in Depression Testing

Yoshikawa and colleagues (Yoshikawa T, Watanabe A, Ishitsuka Y, Nakaya A, Nakatani N. Genome Res. 12:357-366, 2002) measured a difference in baseline immobility in both the forced-swim and tail-suspension tests between C57BL/6 and C3H/He lines. They measured immobilities in F2 mice produced by crossing the 2 parental lines. Mapping analysis of the data revealed 5 genes (chromosomes 6, 8, 8, 11, 17) for the forced-swim phenotype, and 4 (chromosomes 4, 8, 11, 14) for the tail suspension-phenotype. One of the chromosome 8 genes and the chromosome 11 gene apparently represent the same genes for the 2 tests. The chromosome 11 gene mapped near a cluster of GABAA receptor subunit genes, suggesting that a variation in the activity of one of these contributes to the immobility phenotypes; further studies are required to test this hypothesis.

The aforementioned mouse behavioral data and genetic studies indicate that multiple genes contribute in an ensemble fashion to modulate mood. Genetic mapping studies suggest the presence and importance of modifier genes whose protein products may be components of monoaminergic signaling pathways (e.g. receptors, G proteins, kinases) or can influence these cascades. Alternatively, cellular mechanisms and neurotransmitter systems falling outside of the canonical pathways may factor into depressive illness.

Molecular targets in these newly discovered pathways provide opportunities for therapeutic intervention. For example, several pharmaceutical efforts aimed at creating novel antidepressant medications have focused on developing antagonists of two neuropeptide transmitters, corticotropin releasing factor and Substance P. (Keller M, et al., Biol Psychiatry. 2006 Feb. 1; 59(3):216-23. Epub 2005 Oct. 24). For a review of CRF-receptor antagonists, see, e.g., Valdez G R., CNS Drugs. 2006; 20(11):887-96). Thus far, neither has been shown to be clinically effective.

### Kinases and Kinase Inhibitors in Depression

The present invention comprises identification of cellular components, genotypes and gene expression profiles associated with mood disorders. We have determined that there are significant differences in the response phenotypes of various mouse strains in behavioral despair tests that serve as animal models of depression. We have used the differences to identify genes whose activities contribute to the relative depressed/antidepressed status of mice. We studied mouse lines that differ in their immobility responses in despair tests. Time of immobility was measured in both the forced-swim test and tail-suspension test for C57BL/6J and DBA/2J mice. We then examined the concentrations of brain mRNAs from the 2 strains of mice so as to identify those mRNAs whose accumulation could account for the behavioral differences. These data indicate that the ribosomal protein S6 is more highly expressed in brains of C57BL/6J mice than brains of DBA/2J mice. In brain, ribosomal protein S6 can be phosphorylated by ribosomal S6 kinases (RSKs).

Protein kinases are enzymes that catalyze the phosphorylation of hydroxyl groups on tyrosine, serine and threonine residues of proteins. See, for example, Hardie, G. and Hanks, S. (1995) The Protein Kinase Facts Book, I and II, Academic Press, San Diego, Calif.; Stover, D. R. et al., Current Opin in Drug Discovery, (1999) 2(4), 274 285; Adams, J. L., Current Opin. in Drug Discovery, (1999) 2(2), 96 109; and Lawrence D. S. et al., Pharmacol. Ther. (1998) 77(2), 81 114. By doing so, protein kinases mediate virtually all aspects of cell life including cell growth, cell differentiation and cell proliferation. In this regard, abnormal activity of protein kinases has been associated with a host of diseases or medical disorders, ranging from relatively non-life threatening diseases such as psoriasis to extremely virulent diseases such as glioblastoma (brain cancer). See, for example, Levitzki, A. et al., Science, (1995) 267, 1782 1788.

Ribosomal S6 kinases have diverse functions. They are important players in cell cycle progression, cell survival, and cytostatic-factor arrest. Additional roles of RSKs include the feedback inhibition of the Ras-ERK pathway and the regulation of protein synthesis by phosphorylation of polyribosomal proteins and glycogen synthase kinase-3. The enzyme p90 ribosomal S6 kinase (RSK) is an important downstream effector of mitogen-activated protein kinase, but its biological functions are not well understood and it has been minimally studied for its role in the brain where it is known to be expressed.

### Kinase Inhibitors

### Botanical Extracts

In some embodiments, a kinase inhibitor of the present invention comprises a botanical extract. For example, a plant derived flavone, (kaempferol) was recently shown to be an inhibitor of the RSK2, which is known to be present in brain as well as other tissues. See, e.g., US 2005/0233985, which is incorporated herein by reference in its entirety. We have determined that kaempferol will reduce immobility time in C57BL/6J mice in the forced-swim test in a dose dependent manner within minutes after intraperitoneal injection. This response in this behavioral test has been employed to predict the potency of an antidepressant effect. Because the same protein, RPS6, can also regulate other mood-related neurotransmitter-related proteins such as receptors for glutamate and serotonin, modulation of its phosphorylation may have implications for treatment for anxiety as well as depression.

In addition to kaempferol, numerous other kinase inhibitors have been identified and produced, e.g., by organic synthesis or by isolation from natural sources (e.g., plant sources). See, e.g., U.S. Pat. Nos. 7,173,039 and 5,461,146, U.S. Patent Publications 20050233985 and 20060079494, PCT Publication WO 2006/086103A2, each of which is incorporated by reference in its entirety for all purposes.

### Small Molecules

In some embodiments, a kinase inhibitor of the present invention comprises a small molecule. Small molecules extracted from natural sources (e.g., botanical extracts as described above) or created synthetically can function as kinase inhibitors. For example, U.S. Patent Publications 20050233985 and 20060079494, and PCT Publication No. WO 2006/086103 (each of which is incorporated herein by reference) provide a number of small molecule RSK inhibitors. Methods for screening small molecule libraries for kinase inhibition activity are known in the art. In addition to kinase assays such as those provided herein as examples, see, e.g., U.S. Pat. No. 7,202,033, which is incorporated herein by reference.

### Interference and Antisense RNA Inhibitors

In some embodiments, a kinase inhibitor of the present invention comprises an oligonucleotide agent. In such embodiments, a treatment method, for example, comprises the steps of administering to a patient a RSK specific inhibitory composition comprising an anti-sense oligonucleotide or interfering oligonucleotide directed against an RSK, e.g., RSK1, RSK2, RSK3 or RSK4. The ability to specifically inhibit gene function in a variety of organisms utilizing antisense RNA or ds RNA-mediated interference is well known in the fields of molecular biology (see for example C. P. Hunter, Current Biology [1999] 9:R440-442; Hamilton et al., [1999] Science, 286:950-952; and S. W. Ding, Current Opinions in Biotechnology [2000] 11:152-156, hereby incorporated by reference in their entireties).

Interfering oligonucleotides include RNA interference molecules (RNAi)s as well as the DNA sequences encoding for such RNAi. RNAi in mammalian systems includes the presence of short interfering RNA (siRNA) or short hairpin RNA (shRNA). siRNA typically consists of 19-22 nt double-stranded RNA molecules, whereas shRNA typically consists of 19-29 nt palindromic sequences connected by loop sequences, that mimic the structures of micro RNAi. However, larger or smaller nucleic acid sequences than the ranges cited above can be used for the siRNA and shRNA constructs. While not limiting such embodiments of the invention to any particular mechanism of action, down regulation of gene expression is believed to be achieved in a sequence-specific manner by pairing between homologous RNAi sequences and the target RNA. In one embodiment an siRNA construct is prepared comprising a dsRNA that further comprises a 3′ two nucleotide overhang off the sense and antisense strands as described in Elbashir and Tuschl (2001). Genes & Dev. 15: 188-200.

siRNA and shRNA can be introduced into target cells using standard nucleic acid constructs and techniques known to those skilled in the art. For example, a stable system for expressing siRNA or shRNA has been previously described and utilized to generate transgenic animals (Hasuwa et al. FEBS Lett 532, 227-30 (2002), Rubinson et al. Nat Genet. 33, 401-6 (2003) and Carmell et al. Nat Struct Biol 10, 91-2 (2003)). Furthermore, numerous resources are available that describe the design and optimization of RNAi constructs and their use. For example see U.S. Pat. No. 6,506,559 (the disclosure of which is incorporated herein by reference), or the Tushl lab (Rockerfeller University) website for the “siRNA user guide”, located at www.mpibpc.gwdg.de/abteilungen/100/105/sirna.html, or information provided by Ambion, Inc. (2130 Woodward, Austin, Tex. 78744-1832, USA), Sirna Therapeutics (2950 Wilderness Place, Boulder, Colo. 80301), or RNA-TEC NV (Provisorium 2, Minderbroedersstraat 17-19, B-3000 Leuven, Belgium). Expression Cassettes Kits for expression of RNAi sequences in cells are commercially available from Ambion, Inc.

Advantageously, the use of antisense and interference RNAs allows for the design of antisense or interference RNAs that are specific for the individual RSK isotypes. Programs are publicly available for selecting siRNA sequences from a known target gene sequence and thus the design of isotype-specific RNAi sequences is well within the skill of the ordinary practitioner once the target sequence is identified. For example, see Dharmacon, Inc., 1376 Miners Drive #101, Lafayette, Colo. 80026, at the Dharmacon siDESIGN Center at www.dharmacon.com.

Oligonucleotide inhibitors may also comprise aptamers. Aptamers are generally nucleic acid (or peptide) species that have been engineered through repeated rounds of in vitro selection (e.g., SELEX or systematic evolution of ligands by exponential enrichment), to bind specifically to a particular molecular target, such as a small molecule, protein, nucleic acid, and even cells, tissues and organisms. See, e.g., Ellington A D and Szostak J W, Nature, 1990 Aug. 30; 346(6287):818-22, and Bock L C, et al., Nature, 1992 Feb. 6, 355(6360):564-6. Peptide aptamers also find use with the present invention. See, e.g., Hoppe-Seyler F, and Butz K, J Mol. Med. 2000; 78(8):426-30.

### Antibody Inhibitors

In some embodiments, a kinase inhibitor of the present invention comprises an antibody that is specific for RSK. In accordance with one embodiment, an RSK-specific inhibitory composition comprises an antibody that is specific for RSK, and in one embodiment, the antibody is specific for an RSK isotype selected from the group consisting of RSK1, RSK2, RSK3 and RSK4. In accordance with one embodiment, an RSK specific antibody is directed against the adenosine interacting loop of an RSK enzyme, including for example, the AIL of a RSK-isotype selected from the group consisting of RSK1, RSK2, RSK3 and RSK4. Antibodies suitable for use as RSK specific inhibitory compounds include both monoclonal and polyclonal antibodies as well as recombinant proteins comprising the binding domains, as wells as fragments, including Fab, Fab′, F(ab).sub.2, and F(ab′).sub.2 fragments.

### Peptide Inhibitors

In some embodiments, a kinase inhibitor of the present invention comprises a peptide agent. For example, in some embodiments, a protein kinase comprises a hydrophobic pocket (e.g., in the position equivalent to the hydrophobic pocket of mouse Protein Kinase A (PKA) that is defined by residues including Lys76, Leu116, Val80 and/or Lys111 of full-length mouse PKA) and a peptide modulates the protein kinase activity by interaction with the hydrophobic pocket. In such embodiments, a treatment method, for example, comprises the steps of administering to a patient an RSK specific inhibiting composition comprising a peptide directed against an RSK, e.g., RSK1, RSK2, RSK3 or RSK4. The ability to specifically inhibit protein kinases through the use of peptide agents, and methods for selecting peptides and other compounds that modulate protein kinase activity are disclosed, e.g., in US Patent Pub. No. 20080009025 to Alessi, et al., which is incorporated herein by reference.

In addition to the kinase inhibitors discussed above, there are numerous other protein kinase inhibitors, including RSK2 inhibitors, known in the art. See, for example: Cohen M S., et al., Nat Chem. Biol. 2007 March; 3(3):156-60; Sapkota G P, et al., Biochem J. 2007 Jan. 1; 401(1):29-38; and Cohen M S, et al., Science. 2005 May 27; 308(5726):1318-21, each of which is incorporated herein by reference in its entirety.

### Treatment Methods and Compositions

In some embodiments, the present invention provides a composition comprising a kinase inhibitor for the treatment or prevention of depression and/or anxiety. In some preferred embodiments, the kinase inhibitor comprises an RSK inhibitor. In particularly preferred embodiments, the kinase inhibitor is a specific inhibitor of RSK.

In some embodiments, the present inventions comprise a method of treating a cell with a composition comprising an RSK inhibitor composition as described herein, comprising preparing a composition as described herein, and exposing the cell to the composition. In some preferred embodiments, the exposing of the cell occurs in vivo, e.g., in a patient or subject.

In some embodiments, the present invention comprises a method of treating a human or animal disease, comprising administering a therapeutically effective amount of a composition comprising an RSK inhibitor as described herein, and exposing the composition to a human or animal in need thereof, such that the composition is delivered to the human or animal patient.

In some embodiments, the present invention comprises a method of treating a human or animal disease, comprising administering a therapeutically effective amount of a composition comprising a liposome or complex comprising an RSK inhibitor as described herein, and exposing the composition to a human or animal in need thereof such that the active agent is delivered to the human or animal patient.

The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. As used herein, the term “topically” refers to application of the compositions of the present invention to the surface of the skin and mucosal cells and tissues (e.g., alveolar, buccal, lingual, masticatory, or nasal mucosa, and other tissues and cells that line hollow organs or body cavities). Compositions and formulations comprising an RSK inhibitor are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

In some embodiments, a composition according to the present invention is encapsulated in a capsule. In preferred embodiments, the capsule comprises an enteric coating. “Enteric” refers to the small intestine, therefore “enteric coating” generally refers to a coating that substantially prevents release of a medication before it reaches the small intestine. While not limiting the invention to any particular mechanism of action, it is understood that most enteric coatings work by presenting a surface that is stable at acidic pH but breaks down rapidly at higher pH.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Thus, in some embodiments, pharmaceutical compositions of the present invention include, but are not limited to, solutions, emulsions, liposome-containing formulations, and complexes configured, e.g., to improve solubility and/or reduce toxicity. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

Thus, in some embodiments, the compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

In some embodiments of the methods and compositions of the present invention, the composition is in lyophilized form. In some embodiments, the lyophilized composition further comprises a cryoprotectant such as one or more sugars. In preferred embodiments, the cryoprotectant is a sugar selected from a group consisting of trehalose, maltose, lactose, sucrose, and dextran.

In some embodiments, the invention provides mixtures of two or more kinase inhibitors. In preferred embodiments, a mixture of kinase inhibitors comprises an RSK inhibitor. In particularly preferred embodiments, a mixture of kinase inhibitors comprises a kinase inhibitor that is specific for RSK.

In some embodiments, the invention provide pharmaceutical compositions containing (a) a kinase inhibitor (e.g., an RSK inhibitor) and (b) one or more other agents (e.g., depression and/or anxiety therapeutics). Examples of such depression and anxiety therapeutic agents are described above. In some embodiments, two or more combined agents (e.g., depression and/or anxiety therapeutics) may be used together or sequentially.

The present invention also includes methods involving co-administration of compounds comprising a kinase inhibitor (e.g., an RSK inhibitor) described herein with one or more additional active agents (e.g., antidepressants, etc.). Indeed, it is a further aspect of this invention to provide methods for enhancing prior art therapies and/or pharmaceutical compositions by co-administering a composition comprising a kinase inhibitor (e.g., an RSK inhibitor) of this invention. In co-administration procedures, the agents may be administered concurrently or sequentially. In one embodiment, the compounds described herein are administered prior to the other active agent(s). The pharmaceutical formulations and modes of administration may be any of those described above. In addition, the two or more co-administered agents may each be administered using different modes or different formulations. The additional agents to be co-administered can be any of the well-known agents in the art, including, but not limited to, those that are currently in clinical use.

In cases where compounds are sufficiently basic or acidic to form acid or base salts, use of the compounds as salts may be appropriate. Examples of acceptable salts are organic acid addition salts formed with acids which form a physiological acceptable anion, for example, tosylate, methanesulfonate, acetate, citrate, malonate, tartarate, succinate, benzoate, ascorbate, a-ketoglutarate, and a-glycerophosphate. Suitable inorganic salts may also be formed, including hydrochloride, sulfate, nitrate, bicarbonate, and carbonate salts.

Also included within the scope of this invention are the pharmaceutically-acceptable salts formed with non-toxic acids. These acid addition salts include salts derived from inorganic acids such as: hydrochloric acid, nitric acid, phosphoric acid, sulfuric acid, hydrobromic acid, hydriodic acid, nitrous acid, phosphorous acid and the like, as well as salts of non-toxic organic acids including aliphatic mono and dicarboxylates, phenyl-substituted alkanoates, hydroxy alkanoates and alkanedioates, aromatic acids, aliphatic and aromatic sulfonic acids etc. Such pharmaceutically-acceptable salts thus include (but are not limited to): tosylate, methanesulfonate, acetate, citrate, malonate, tartarate, succinate, benzoate, ascorbate, a-ketoglutarate, and a-glycerophosphate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, fluorodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, benzenesulfonates, toluenesulfonate, chlorobenzenesulfonate, xylenesulfonate, phenylacetate, phenylpropionate, phenylbutyrate, citrate, lactate, .beta.-hydroxybutyrate, glycollate, malate, tartrate, methanesulfonate, propanesulfonates, naphthalene-1-sulfonate, naphthalene-2-sulfonate, mandelate and the like salts.

Uses of the compositions and methods provided by the present invention encompass human and non-human subjects and samples from those subjects, and also encompass research as well as diagnostic applications. Thus, it is not intended that the present invention be limited to any particular subject and/or application setting.

### Diagnostic and Screening Methods and Compositions

In some embodiments, the present invention provides methods for the detection of depression and/or anxiety, or the risk of developing depression of anxiety. In some embodiments, the present invention provides a method comprising providing a sample from a patient suspected of having or being at risk of depression and/or anxiety and determining an activity for Ribosomal Protein S6. In some embodiments, the present invention comprises determining a concentration Ribosomal Protein S6 while in some embodiments, it comprises determining a feature of nucleic acid encoding ribosomal protein S6. For example, the presence or absence of a particular nucleotide sequence (e.g., a mutation or polymorphism, including but not limited to a single nucleotide polymorphism), associated with expression or function (e.g., transcription, translation, protein function, etc.) of ribosomal protein S6 may be determined as an aspect of the present invention. Sequences need not be in the coding region of a ribosomal protein S6 gene, however. It is contemplated that polymorphisms in non-coding regions (e.g., flanking regions, introns, etc.) associated with ribosomal protein S6 function find use in the present invention.

In some embodiments, method of the present invention comprises determining a protein kinase activity as a step in determining the presence of or risk of depression or anxiety in a subject. In preferred embodiments, the method comprises providing a sample from a patient suspected of having or being at risk of depression and/or anxiety and determining an activity for a protein kinase. In some preferred embodiments, the method of the present invention comprises determining an activity of an RSK. In particularly preferred embodiments, the method of the present invention comprises providing a sample from a subject and measuring the concentration and/or activity of p90 Ribosomal Protein Kinase as a diagnostic step in detecting depression or anxiety, or risk therefore, in said subject. In some embodiments, the method of the present invention comprises examining nucleic acid encoding p90 ribosomal protein kinase. For example, the presence or absence of a particular nucleotide sequence (e.g., a mutation or polymorphism, including but not limited to a single nucleotide polymorphism), associated with expression or function (e.g., transcription, translation, protein function, etc.) of p90 ribosomal protein kinase may be determined as an aspect of the present invention. As noted above, sequences need not be in the coding region of a p90 ribosomal protein kinase gene. It is contemplated that polymorphisms in non-coding regions (e.g., flanking regions, introns, etc.) associated with p90 ribosomal protein kinase function find use in the present invention.

In some embodiments, the present invention provides methods of tailoring treatments to the biochemical status of a subject or patient. It is contemplated that features of ribosomal protein S6 and/or p90 ribosomal protein kinase may be altered by particular biochemical circumstances of a subject or patient, including but not limited to the presence of other drugs or medications (e.g. for treatment of depression or unrelated conditions), or biochemical changes caused by other circumstances. The present invention provides methods for determining features ribosomal protein S6 and/or p90 ribosomal protein kinase as described herein, which find use for monitoring the effect of the biochemical status of the subject on ribosomal protein S6 and/or p90 ribosomal protein kinase. In some embodiments, therapy for depression and/or anxiety is selected, adjusted, or altered accordingly.

Typically, a biological sample used for measuring the RSK quantification factor will comprise a tissue or cell sample recovered from an individual, for example during a biopsy. However, blood or serum samples can also be screened for the presence of RSK nucleic acid sequences or peptides. Analysis of the biological sample to quantitate the RSK content of the sample can be conducted using standard techniques known to those skilled in the art. Over-expression of RSK can be detected, e.g., by determining cellular nucleic acid concentrations (e.g., mRNA levels) or by determining cellular RSK protein concentrations. This includes the use of in situ analysis as well as the purification of the RSK nucleic acid or protein. For example, the amount of RSK nucleic acid present in the sample can be determined using labeled complementary RSK nucleic acid sequences and standard Southern or Northern blotting techniques or in situ hybridization techniques. In other embodiments, RSK nucleic acid is detected using any of the nucleic acid molecular methods known to those of skill in the art, including but not limited to polymerase chain reaction, Invader assay, ligase chain reaction, etc.

Similarly, the amount of RSK protein present in the sample can be determined using antibodies that are specific for RSK epitopes or using other analytical techniques. Alternatively, in one embodiment the RSK protein is purified from the biological sample, and the RSK kinase activity of the recovered material is determined through the use of an in vitro kinase assay. In some embodiments, the kinase activity is measured using phosphospecific and/or nonphosphospecific antibodies directed against a RSK substrate after conducting a kinase assay.

In some embodiments, RSK activity is determined by isolating RSK protein from the biological samples, conducting in vitro kinase assays and determining the rate of formation of phosphorylated substrate. In some embodiments, the amount of RSK protein is determined by contacting the RSK protein with a labeled antibody specific for RSK protein, removing the non-bound and non-specific antibody; and quantifying the amount of label remaining to determine the amount of RSK protein present. In further embodiments, the amount of RSK nucleic acids present in the biological sample is determined by contacting the nucleic acids of the sample with a labeled RSK complementary nucleic acid probe, removing the non-bound and non-specific probe; and quantifying the amount of label remaining to determine the amount of RSK nucleic acid. Generally, the amount of RSK detected in a biological sample is measured against an internal or external standard. For example, when an internal standard is used to determine if the RSK nucleic acid or protein is being over- or under expressed, the levels detected in the recovered biological sample can be compared to the nucleic acid or protein levels of a non-RSK gene that is expressed in the same tissue used for measuring the RSK levels. In some embodiments a house keeping gene is selected as the internal reference, including for example, actin, Ran or some other gene whose level typically does not fluctuate in the cells selected for the biological sample (i.e., the biopsy tissue). Similarly when measuring the activity of RSK, the detected RSK activity in the tissue can be compared to another enzymatic activity present in the same tissue used for measuring the RSK activity.

In some embodiments, RSK levels/activity measured in the biological sample recovered from a subject to be screened are compared to an external standard (e.g., a biological sample derived from another source). In some embodiments, the external standard constitutes an average of RSK levels/activity measured from one or more healthy individuals and used to establish a baseline of RSK activity. Standard curves utilized are based on the tissue type (e.g., brain tissue) and amounts of starting material used. Such standard curves will be used to determine if an individual's RSK levels are higher than the population's average levels.

In some embodiments, a diagnostic kit for detecting the presence or risk of depression and/or anxiety is provided. The kit comprises reagents for detecting and quantitating an amount of RSK, a level of expression of a gene encoding an RSK, or RSK activity present in a biological sample. In some embodiments, a kit of the present invention comprises as RSK quantifying agent selected from the group consisting of as RSK specific antibody, a nucleic acid sequence complementary to as RSK gene sequence or mRNA, or as RSK substrate and reagents for conducting in vitro kinase assays. In some embodiments, the antibodies or nucleic acids provided with the kit are labeled, or reagents are provided for labeling the RSK-specific antibodies or nucleic acid sequences. Reagents can be packaged in a variety of containers, e.g., vials, tubes, bottles, and the like. In some embodiments, one or more reagents are provided in a dry or lyophilized form. Other reagents can be included in separate containers and provided with the kit; e.g., positive control samples, negative control samples, buffers, etc. In some preferred embodiments, a kit comprises instructional materials for using the reagents to quantitate an RSK factor or activity.

In some embodiments, the present invention proves a kit for screening compounds for anxiolytic and/or antidepressant activity. In preferred embodiments, a kit for screening compounds for anxiolytic and/or antidepressant activity comprises reagents for testing a compound for kinase inhibitor activity, using e.g., an activity assay as described above. In preferred embodiments, a kit of the present invention comprises one or more reagents for screening a test compound for RSK inhibition. In particularly preferred embodiments, the present invention provides a kit comprising one or more reagents for screening a test compound for specific inhibition of RSK.

Methods of testing kinase and kinase inhibition activity are well known in the art. See, e.g., US Patent Pub. No. 2005/0233985. By way of example and not by way of limitation, in some kinase assays, a Glutathione-S-transferase (GST)-fusion protein containing the sequence—RRRLASTNDKG (for serine/threonine kinase assays) or -VSVSETDDYAEIIDEEDTFT (for tyrosine kinase assays) is adsorbed in one or more test wells (e.g., using MAXISORP surface treatment). The wells are blocked with sterile 3% tryptone in phosphate buffered saline and can be stored at 4° C. for up to 6 months. Kinase (5 nM) in 70 μl of kinase buffer (5 mM 25-glycerophosphate pH 7.4, mM HEPES pH 7.4, 1.5 mM DTT, 30 mM MgCl.sub.2, 0.15 M NaCl) is dispensed into each well. A test compound is added, and reactions are initiated by the addition of 30 μl of ATP, for a final ATP concentration of 10 μM, unless indicated otherwise. Reactions are terminated after 10 to 45 min by addition of 75 μl of 500 mM EDTA, pH 7.5. Typically, assays measure the initial velocity of reaction. After extensive washing of the wells, polyclonal phosphospecific antibody developed against the phosphopeptide and HRP-conjugated anti-rabbit antibody (211-035-109, Jackson ImmunoResearch Laboratories) is used to detect serine phosphorylation of the substrate. HRP-conjugated anti-phospho-tyrosine antibody (RC20, BD Transduction Laboratories) is used for phospho-tyrosine detection.

### EXPERIMENTAL

The following example is provided in order to demonstrate and further illustrate certain preferred embodiments and aspects of the present invention and are not to be construed as limiting the scope thereof.

### Example 1

### Identification of Cellular Functions Associated with Depression

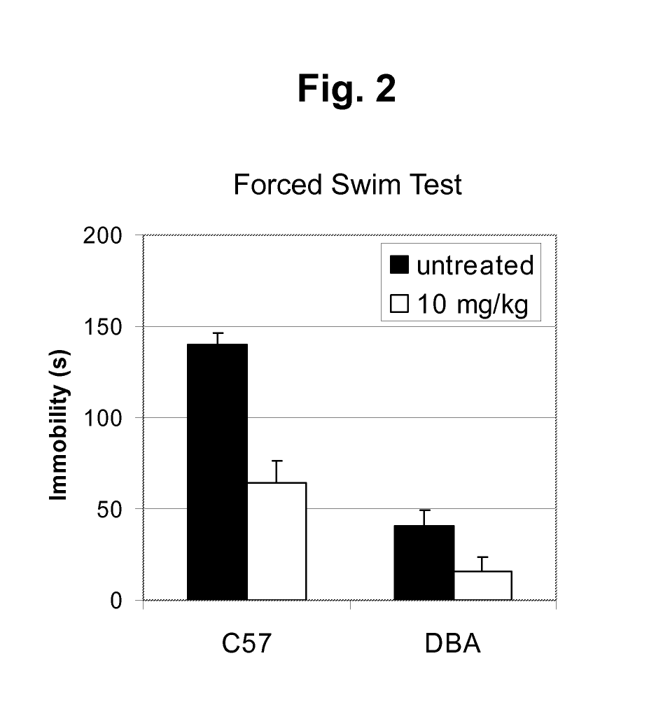

As described above, the murine responses in forced swim and tail suspension tests provides a model for determining depression. We tested C57BL/6J and DBA/2J mice in the forced-swim and tail-suspension tests. In the forced-swim test, mice were gently placed in a clear plastic cylinder, diameter of 16 cm, height 25 cm, filled with 10 cm of clear water at 25° C. Test duration was 6 minutes and immobility, defined as the absence of all movement except minor movement required for the mouse to keep its head above the surface, was measured during the last 4 minutes. In the tail-suspension test, mice were suspended from a metal rod mounted 50 cm above the surface by fastening the tail to the rod with adhesive tape. The duration of the test was 6 minutes and immobility, defined as the absence of all but respiratory movements, was measured during the last 4 minutes. During the last 4 minutes of a 6-minute forced swim, unmedicated inbred C57BL/6J mice spent 152 seconds of the possible 240 seconds immobile (FIG. 2). In contrast, inbred DBA/2J mice had baseline (unmedicated) immobilities of 47 seconds (see FIG. 2). This indicates that genetic differences between the two mouse strains influence behaviors in the test. We investigated the hypothesis that the differences between these two inbred mouse lines that result in differing baseline immobilities in the forced-swim test at least in part arise from differences in the amount of mRNA that accumulates from specific genes.

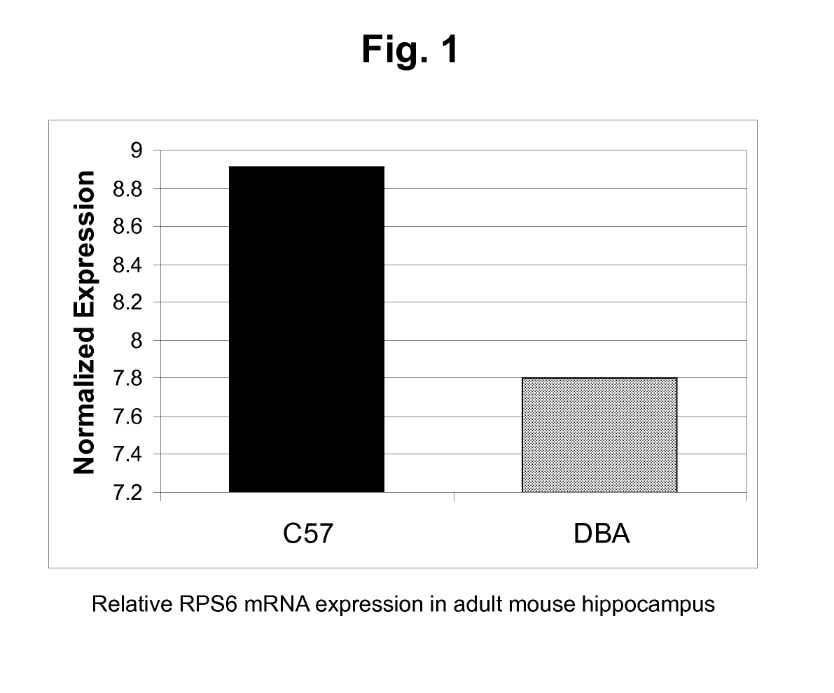

The concentrations of each of more than 20,000 mRNAs in the brains of these mouse strains are available in public databases compiled at http://www.GeneNetwork.org. Comparing the concentrations of brain mRNAs found the strains of mice described above, we identified a single gene on chromosome 4 whose output mRNA concentration significantly differed between the brains of C57BL/6J and DBA/2J mice (FIG. 1). The chromosome 4 gene (mouse gene CAA90936, human homologue gene AAH09427) encodes a previously known protein, ribosomal protein S6 (rpS6), whose activity is known to be regulated by a specific activating protein, p90 ribosomal S6 kinase, or RSK (mouse protein NP_{—}033123, human protein NP_{—}002944) (Hayashi M, Fearns C, Eliceiri B, Yang Y, Lee J D. Cancer Res. 65:7699-7706, 2005). Because the rpS6 mRNA has a higher concentration in C57BL/6J mice than in DBA/2J mice, and the C57BL/6J mice had much higher immobility scores than did the DBA/2J mice, we reasoned that a compound that inhibited the activity of RSK would alter behavior in the behavioral tests in the manner of antidepressant compounds.

A compound that inhibits the activity of this kinase in cultured tumor cells, 3Ac-SL0101, has recently been synthesized (D. E. Clark, T. M. Errington, J. A. Smith, H. F. Frierson, Jr., M. J. Weber, D. A. Lannigan. Cancer Research 65, 3108-3116, 2005, US Patent Publication 20050233985, see also United States Patent Application 20060079494 to Santi, et al., each of which is incorporated herein by reference in its entirety).

Exemplary methods of synthesizing RSK inhibitors are provided, e.g., in WO06086103A2 to Hecht, et al., which is incorporated herein by reference in its entirety.

We purchased some of the compound (Toronto Research Chemicals Inc., 2 Brisbane Rd., North York, Ontario, Canada M3J 2J8) and dissolved it in phosphate-buffered saline (PBS) containing 2.5 percent methanol.

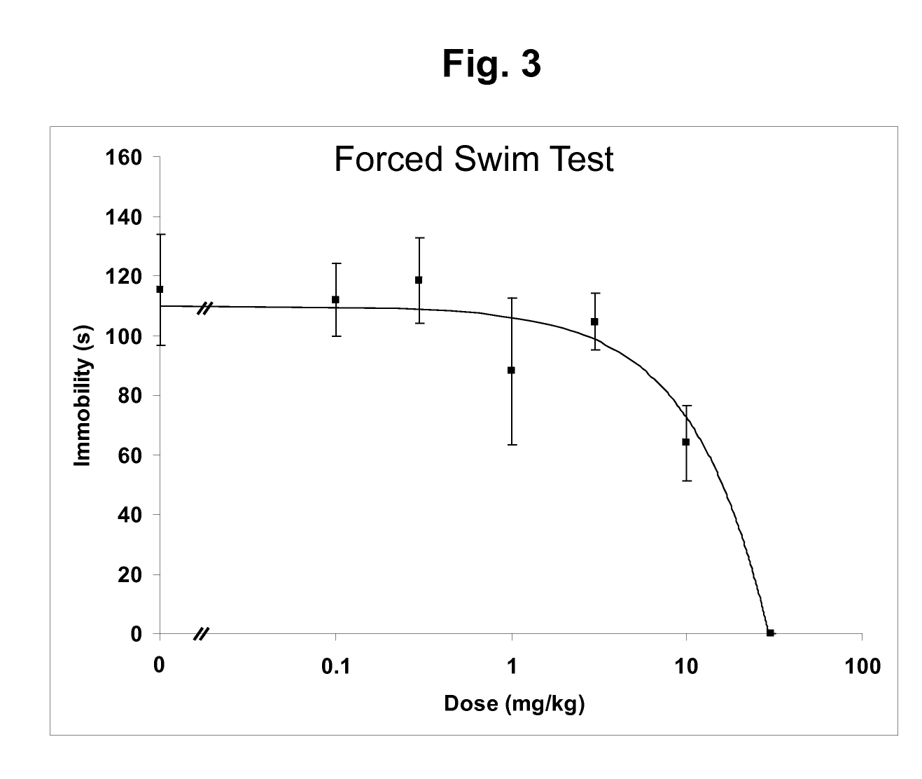

When plain PBS/methanol solution (without 3Ac-SL0101) was administered to C57BL/6J mice, their immobility was 127 seconds. When the solution containing the 3Ac-SL0101 compound was administered in a very low dose, their immobility was reduced to 78 seconds, indicative of an antidepressant-like effect. A 20-fold higher dose reduced immobility to 0 seconds. Subsequent tests revealed a monotonic dose-response effect (FIG. 3), and also showed that the compound reduced immobility in the tail-suspension test. When the compound was administered to DBA/2J mice, it was effective at an approximately 10-fold lower dose than in C57BL/6J mice, highly consistent with the notion that it is functioning by reducing the activity of rpS6.

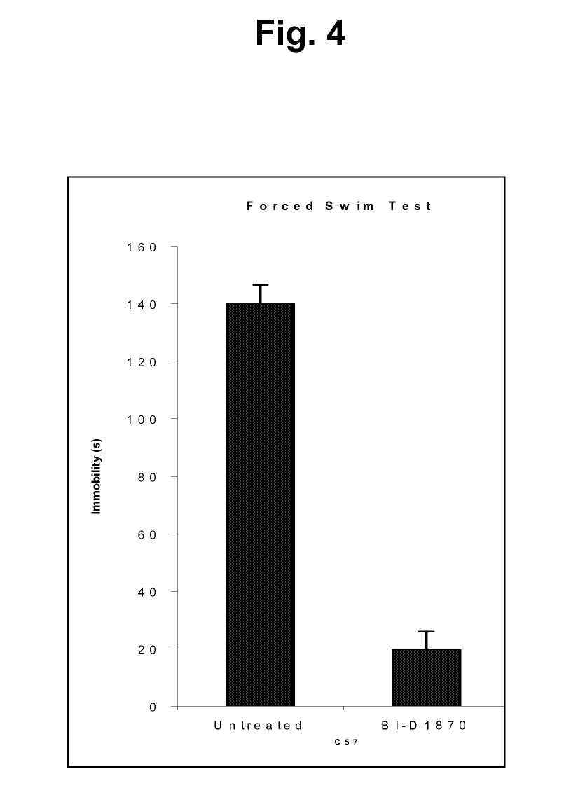

The compound B1-D1870 is known to be a specific inhibitor of the p90 RSK (see, e.g., Biochem. J. (2007) 401, 29-38, incorporated herein by reference). BI-D1870 (purchased from Division of Signal Transduction Therapy, College Of Life Sciences, University of Dundee, Dow Street, Dundee DD1 5EH, Scotland, U.K.) was prepared and tested as described above for 3Ac-SL0101, with the results shown in FIG. 4. These data show that, as seen with 3Ac-SL0101, treatment of mice with this compound produced a substantial reduction the time of immobility in the forced swim test, indicative of an antidepressant-like effect.

These data, along with the literature that well supports that these animal tests are reflective of human depression, indicate that these and similar compound (see, e.g., U.S. Patent Publication 20050233985), can be considered as a lead compounds for testing as antidepressants and/or anxiolytics.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

1. A method of treating or preventing depression or anxiety in a subject comprising administering a pharmaceutical preparation comprising a therapeutic agent that alters the state of ribosomal protein S6 in a cell in said subject.

2. (canceled)

3. A method of treating or preventing depression or anxiety in a subject comprising administering a pharmaceutical preparation comprising a therapeutic agent that alters the activity of a protein kinase in a cell in said subject.

4. The method of claim 2, wherein said altering the activity of said protein kinase comprises inhibiting said protein kinase.

5. The method of claim 4, wherein said protein kinase is p90 Ribosomal S6 Kinase.

6. (canceled)

7. The method of claim 5, wherein said therapeutic agent comprises a specific inhibitor of p90 Ribosomal S6 Kinase.

8. The method of claim 1, wherein said therapeutic agent comprises an and agent selected from the group consisting of and antisense oligonucleotide, an interfering RNA, an aptamer an antibody, or a flavenoid extract of Forsteronia refracta or Zingiber zerumbet.

9-13. (canceled)

14. The method of claim 8 wherein said therapeutic agent comprises an SL0101 compound or a pharmaceutically acceptable salt or hydrate thereof.

15. The method of claim 14, wherein said SL0101 compound comprises SL0101-1, SL0101-2 or SL0101-3, or 3Ac-SL0101, a chemical derivative, a chemical analog, or a chemical precursor of SL0101, or a pharmaceutically acceptable salt or hydrate thereof.

16. (canceled)

17. The method of claim 1, wherein said therapeutic agent comprises a chemical derivative, a chemical analog, or a chemical precursor of a pteridinone.

18. The method of claim 17, wherein said pteridinone comprises BI-D1870 or a pharmaceutically acceptable salt or hydrate thereof, or a chemical derivative, a chemical analog, or a chemical precursor of a dihydropteridinone.

19. (canceled)

20. The method of claim 1, wherein said pharmaceutical preparation further comprises a second therapeutic agent, wherein said second therapeutic is a known therapeutic agent for treatment of depression and/or anxiety, and wherein said known therapeutic agent is selected from the group consisting of a selective serotonin reuptake inhibitor, a serotonin and norepinephrine reuptake inhibitor, a dopamine reuptake inhibitor, a tetracyclic antidepressant, a combined reuptake inhibitor, a receptor blocker, tricyclic antidepressant, a monoamine oxidase inhibitor, a benzodiazepine, a beta-blocker, and a non-benzodiazepine hypnotic.

21-26. (canceled)

27. The method of claim 1, wherein said administering comprises oral administration, topical administration, transdermal administration, parenteral administration, or pulmonary administration.

28-33. (canceled)

34. A method of selecting a compound for treatment or prevention of depression or anxiety, comprising:
a) selecting a test compound suspected of altering the state of ribosomal protein S6; and
b) determining the ability of said test compound to inhibit despair behavior in a test animal in a despair-inducing test,
wherein inhibition of despair behavior is indicative of efficacy of said test compound in the treatment or prevention of depression or anxiety.

35. The method of claim 34, wherein said test animal is a mouse and wherein said despair-inducing test is selected from the group consisting of a forced swim test and a tail suspension test.

36-37. (canceled)

38. The method of claim 35, wherein said test animal is a member of a strain of recombinant inbred mice, and wherein said method further comprises determining whether said test compound reduces said despair behavior in a dose dependent manner, wherein said dose-dependent manner comprises dose-dependent suppression of immobility in proportion to the expression in the brain of said strain of mice of a protein kinase known to phosphorylate ribosomal protein S6.

39-40. (canceled)

41. A method of screening a compound for use in treatment or prevention of depression or anxiety, comprising determining the effect of said compound on the activity of a p90 ribosomal protein kinase, wherein alteration of the activity of said p90 ribosomal protein kinase is indicative of utility of said compound in treatment or prevention of depression or anxiety, wherein said alteration of the activity of said p90 ribosomal protein kinase comprises inhibition of said kinase, and wherein said determining comprises an in vitro assay for protein kinase activity.

42-45. (canceled)

46. A method of screening a subject for depression or anxiety, comprising:
a) providing a sample from said subject; and
b) determining a feature of ribosomal protein S6 in said sample,
wherein said feature of ribosomal protein S6 is indicative of the presence or risk of developing depression or anxiety.

47. The method of claim 46, wherein said determining the feature of ribosomal protein S6 comprises measuring the concentration of ribosomal protein S6 in said sample.

48. A method of screening a subject for depression or anxiety, comprising:
a) providing a sample from a subject; and
b) determining an activity of p90 ribosomal protein kinase in said sample,
wherein activity of ribosomal protein S6 is indicative of the presence or risk of developing depression or anxiety.

49-69. (canceled)
